(19) United States
(12) Patent Application Publication
(10) Pub. No.: US 2026/0096888 A1
(43) Pub. Date: Apr. 9, 2026

(54) IMPLANTABLE PRESSURE SENSOR

(71) Applicant: Boston Scientific Scimed, Inc., Maple Grove, MN (US)

(72) Inventors: Jean M. Bobgan, Maple Grove, MN (US); Keith R. Maile, New Brighton, MN (US); James J. Scutti, Norwell, MA (US)

(21) Appl. No.: 18/905,814

(22) Filed: Oct. 3, 2024

(51) Int. Cl. A61F 2/26; A61F 2/48; G01L 7/08

(52) U.S. Cl. CPC A61F 2/26; A61F 2/482; G01L 7/082; A61F 2210/0009; A61F 2220/0058

(57) ABSTRACT

A method of making a pressure sensor to be positioned in fluidic connection with a fluid passageway of a housing of an implantable fluid operated device includes: providing a flexible metal diaphragm to a metal housing of the pressure sensor, where the metal housing defines an interior cavity of the metal housing; and attaching the flexible metal diaphragm to the metal housing. The flexible metal diaphragm has a first portion that is unattached to the metal housing and that, when positioned in fluidic connection with the fluid passageway of the housing of the implantable fluid operated device, is configured to move inward and outward with respect to the interior cavity in response to a fluid pressure in the fluid passageway. The first portion of the flexible metal diaphragm has a thickness of less than 40 μm, and characteristic metal grain sizes of the first portion are smaller than 10 μm.

### TECHNICAL FIELD

This disclosure relates generally to bodily implants, and more specifically to bodily implants including an implantable pressure sensor.

### BACKGROUND

Active implantable fluid operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid filled implant components of the device. In some implantable fluid operated devices, an implantable pumping device may be manually operated by the user to provide for the transfer of fluid between a reservoir and the fluid filled implant components of the device. Manipulation of the manually operated implantable pumping device may be challenging for some patients. Further, such manual operation of the pumping device may make it difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Some implantable fluid operated devices may include an electronic control system including an electronically controlled manifold providing for the transfer of fluid within the implantable fluid operated device. The use of the electronic control system may provide for more accurate actuation and control of the flow of fluid between components of the inflatable device, thus improving performance and efficacy of the device, as well as patient comfort and safety. In both a manually operated implantable pumping device and in an electronically controlled pumping device, one or more pressure sensors may be used to monitor pressures of fluids in different parts of the device. The durability and reliability of the device, including the one or more pressure sensors, may be important aspects for the devices.

### SUMMARY

According to a general aspect, an implantable fluid operated device includes: a fluid reservoir; a fluid receiver; and a fluid control system configured to control fluid flow between the fluid reservoir and the fluid receiver. The fluid control system includes: a housing including a fluidic architecture defining one or more fluid passageways within in the housing; at least one pump positioned in fluidic connection with at least one of the one or more fluid passageways, the at least one pump being configured to pump fluid from the fluid reservoir to the fluid receiver; and a pressure sensor positioned in fluidic connection with at least one of the one or more fluid passageways. The pressure sensor includes a metal housing including one or more interior cavities; electrical circuitry configured for converting a pressure into an electrical signal; a flexible metal diaphragm attached to the metal housing and having a first portion positioned between an interior cavity of the one or more interior cavities and a fluid passageway and the first portion being configured to move inward and outward with respect to an interior cavity in response to a fluid pressure in the fluid passageway. The first portion of the flexible metal diaphragm has a thickness of less than 40 μm, and characteristic metal grain sizes of the first portion are smaller than 10 μm.

In some aspects, the metal housing is a titanium housing and the flexible metal diaphragm is a flexible titanium diaphragm.

In some implementations, the flexible metal diaphragm is attached to the metal housing by a welded joint between the flexible metal diaphragm and the metal housing.

In some implementations, the welded joint between the flexible metal diaphragm and the metal housing is made at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, the flexible metal diaphragm is attached to the metal housing by a diffusion bonded joint between the flexible metal diaphragm and the metal housing.

In some implementations, the diffusion bonded joint between the flexible metal diaphragm attached to the metal housing is made at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, the one or more interior cavities include at least one fluid-filled cavity that is fluidically coupled to the flexible metal diaphragm and to the electrical circuitry, wherein the electrical circuitry is configured for converting a displacement of the flexible metal diaphragm into the electrical signal.

In some implementations, the techniques described herein relate to a method of making a pressure sensor to be positioned in fluidic connection with a fluid passageway of a housing of an implantable fluid operated device. The method includes: providing a flexible metal diaphragm to a metal housing of the pressure sensor, where the metal housing defines an interior cavity of the metal housing; and attaching the flexible metal diaphragm to the metal housing. The flexible metal diaphragm has a first portion that is unattached to the metal housing and that, when positioned in fluidic connection with the fluid passageway of the housing of the implantable fluid operated device, is configured to move inward and outward with respect to the interior cavity in response to a fluid pressure in the fluid passageway. The first portion of the flexible metal diaphragm has a thickness of less than 40 μm, and characteristic metal grain sizes of the first portion are smaller than 10 μm.

In some implementations, the metal housing is a titanium housing and the flexible metal diaphragm is a flexible titanium diaphragm.

In some implementations, attaching the flexible metal diaphragm to the metal housing includes welding the flexible metal diaphragm to the metal housing.

In some implementations, welding the flexible metal diaphragm to the metal housing includes welding the flexible metal diaphragm to the metal housing at a second portion of the flexible metal diaphragm that is more than 1 mm away, along a surface of the flexible metal diaphragm, from any part of the first portion of the flexible metal diaphragm.

In some implementations, attaching the flexible metal diaphragm to the metal housing includes diffusion bonding the flexible metal diaphragm to the metal housing.

In some implementations, diffusion bonding the flexible metal diaphragm to the metal housing includes diffusion bonding the flexible metal diaphragm to the metal housing at a second portion of the flexible metal diaphragm that is more than 1 mm away, along a surface of the flexible metal diaphragm, from any part of the first portion of the flexible metal diaphragm.

In some implementations, the techniques described herein relate to an implantable fluid operated device that includes: a fluid reservoir; a fluid receiver; and a fluid control system configured to control fluid flow between the fluid reservoir and the fluid receiver, the fluid control system including: a housing including a fluidic architecture defining one or more fluid passageways within in the housing; at least one pump positioned in fluidic connection with at least one of the one or more fluid passageways, the at least one pump being configured to pump fluid from the fluid reservoir to the fluid receiver; a pressure sensor positioned in fluidic connection with at least one of the one or more fluid passageways, the pressure sensor including: a metal housing including one or more interior cavities; electrical circuitry configured for converting a pressure into an electrical signal; a flexible metal diaphragm attached to the metal housing and having a first portion positioned between an interior cavity of the one or more interior cavities and a fluid passageway and the first portion being configured to move inward and outward with respect to an interior cavity in response to a fluid pressure in the fluid passageway, the first portion of the flexible metal diaphragm having a thickness of less than 40 μm, and characteristic metal grain sizes of the first portion being smaller than 10 μm.

In some implementations, the metal housing is a titanium housing and the flexible metal diaphragm is a flexible titanium diaphragm.

In some implementations, the flexible metal diaphragm is attached to the metal housing by a welded joint between the flexible metal diaphragm and the metal housing.

In some implementations, the welded joint between the flexible metal diaphragm and the metal housing is made at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, the flexible metal diaphragm is attached to the metal housing by a diffusion bonded joint between the flexible metal diaphragm and the metal housing.

In some implementations, the diffusion bonded joint between the flexible metal diaphragm attached to the metal housing is made at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, the thickness of the first portion of the flexible metal diaphragm is less than 25 μm, and metal grain sizes of the first portion are smaller than 6 μm.

In some implementations, the thickness of the first portion of the flexible metal diaphragm is less than 16 μm, and metal grain sizes of the first portion are smaller than 4 μm.

In some implementations, the one or more interior cavities include at least one fluid-filled cavity that is fluidically coupled to the flexible metal diaphragm and to the electrical circuitry, wherein the electrical circuitry is configured for converting a displacement of the flexible metal diaphragm into the electrical signal.

In some implementations, the techniques described herein relate to a method of making a pressure sensor to be positioned in fluidic connection with a fluid passageway of a housing of an implantable fluid operated device. The method includes: providing a flexible metal diaphragm to a metal housing of the pressure sensor, where the metal housing defines an interior cavity of the metal housing; and attaching the flexible metal diaphragm to the metal housing. The flexible metal diaphragm has a first portion that is unattached to the metal housing and that, when positioned in fluidic connection with the fluid passageway of the housing of the implantable fluid operated device, is configured to move inward and outward with respect to the interior cavity in response to a fluid pressure in the fluid passageway. The first portion of the flexible metal diaphragm having a thickness of less than 40 μm, and characteristic metal grain sizes of the first portion being smaller than 10 μm.

In some implementations, the metal housing is a titanium housing and wherein the flexible metal diaphragm is a flexible titanium diaphragm.

In some implementations, attaching the flexible metal diaphragm to the metal housing includes welding the flexible metal diaphragm to the metal housing.

In some implementations, welding the flexible metal diaphragm to the metal housing includes welding the flexible metal diaphragm to the metal housing at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, attaching the flexible metal diaphragm to the metal housing includes diffusion bonding the flexible metal diaphragm to the metal housing.

In some implementations, diffusion bonding the flexible metal diaphragm to the metal housing includes diffusion bonding the flexible metal diaphragm to the metal housing at a second portion of the flexible metal diaphragm that is more than 1 mm away, along a surface of the flexible metal diaphragm, from any part of the first portion of the flexible metal diaphragm.

In some implementations, the second portion of the flexible metal diaphragm is non-parallel to the first portion of the flexible metal diaphragm.

In some implementations, the techniques described herein relate to a method, further including filling the interior cavity with fluid that is fluidically coupled to the flexible metal diaphragm and to electrical circuitry in the pressure sensor.

### BRIEF DESCRIPTION OF THE DRAWINGS

FIG. 1 is a block diagram of an implantable fluid operated inflatable device according to an aspect.

FIG. 2 illustrates a system including an example implantable fluid operated inflatable device according to an aspect.

FIG. 3 is a schematic diagram of a fluidic architecture of an implantable fluid operated inflatable device according to an aspect.

FIG. 4 is a schematic cutaway perspective view of an example pressure sensor.

FIG. 5 is a cross-sectional view of the example pressure sensor of FIG. 4.

FIG. 6 is a schematic cross-sectional view of an example metal housing and an example flexible metal diaphragm of a pressure sensor.

FIG. 7 is a photograph of a flexible titanium diaphragm, seen from the bottom, or exterior, side after the diaphragm has been welded to a bottom face of the metal housing.

FIG. 8 is a microscopic image of a weld joint between a flexible titanium diaphragm and a bottom surface of a perimeter rim of a metal housing.

FIGS. 9 and 10 are enlarged versions of the microscopic image of the weld joint shown in FIG. 8.

FIG. 11 is a schematic cross-sectional view of another example metal housing and an example flexible metal diaphragm of a pressure sensor.

FIG. 12 is a photograph of a flexible titanium diaphragm, seen from the bottom, or exterior, side after the diaphragm has been welded to a flange of a metal housing.

FIG. 13 is a microscopic image of a weld joint between a flexible titanium diaphragm and a flange of a metal housing with perimeter rim at the bottom side of the housing.

FIG. 14 is a flowchart of an example process for making a pressure sensor to be positioned in fluidic connection with a fluid passageway of a housing of an implantable fluid operated device.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

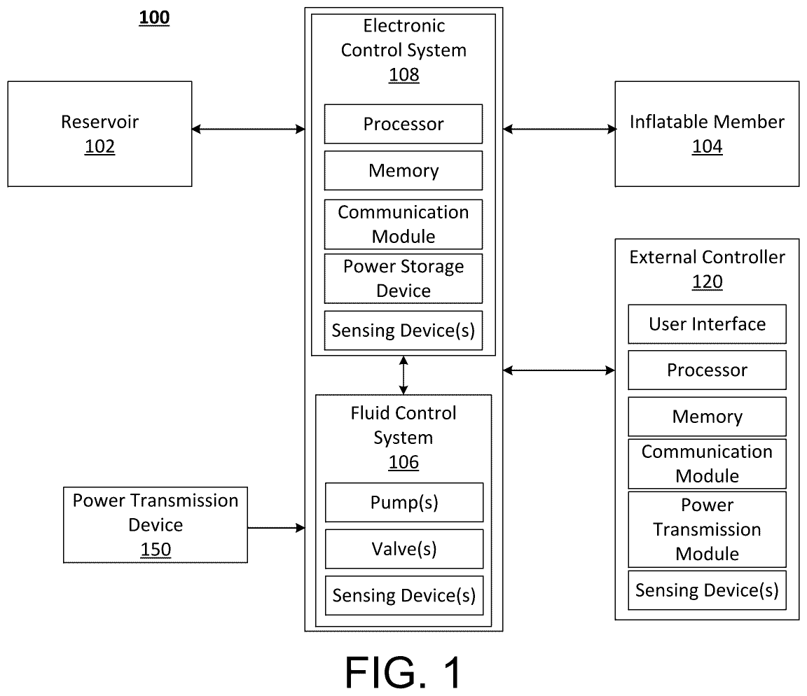

FIG. 1 is a block diagram of an example implantable fluid operated inflatable device 100. The example inflatable device 100 shown in FIG. 1 includes a fluid reservoir 102, a fluid receiver (e.g., an inflatable member) 104, a fluid control system 106, and an electronic control system 108. The electronic control system 108 may interface with the fluid control system 106. The fluid control system 106 can include fluidics components such as one or more pumps, one or more valves and the like configured to transfer fluid between the fluid reservoir 102 and the fluid receiver. The fluid control system 106 can include one or more sensing devices (e.g., pressure sensors, flow rate sensors, thermometers, etc.) that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example, an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in or incorporated into a housing. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable device 100. In some implementations, at least some aspects of the operation of the implantable fluid operated inflatable device 100 may be manually controlled.

In some examples, electronic monitoring and control of the fluid operated inflatable device 100 may provide for improved patient control of the device, improved patient comfort, improved patient safety, and the like. In some examples, electronic monitoring and control of the fluid operated device 100 may afford the opportunity for tailoring of the operation of the inflatable device 100 by a physician without further surgical intervention. Fluidic architecture defining the flow and control of fluid through the fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

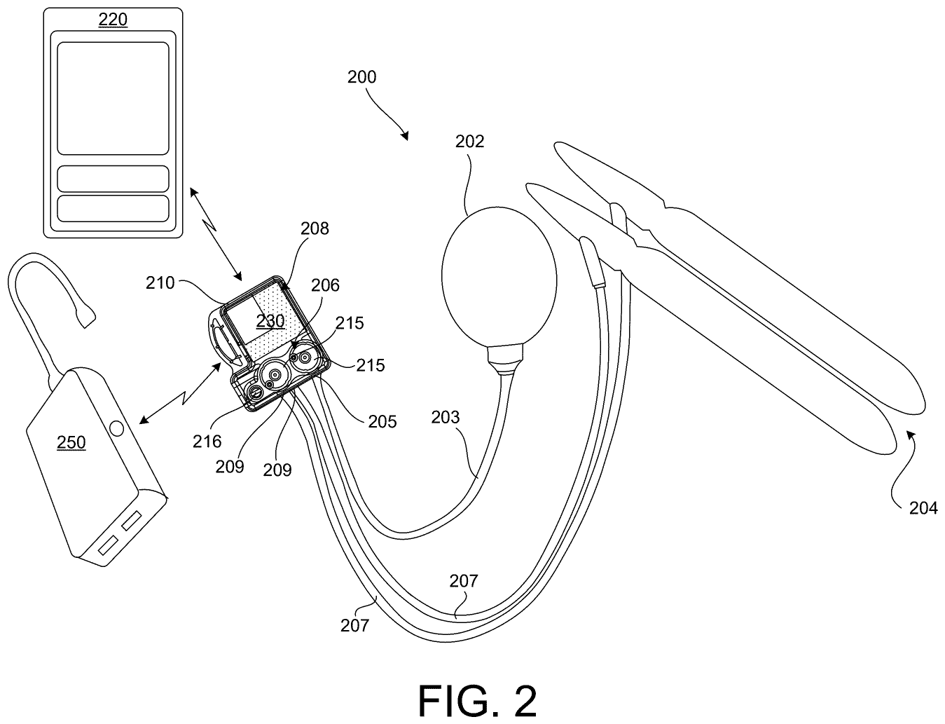

The example implantable fluid operated inflatable device 100 may be representative of a number of different types of implantable fluid operated devices. For example, the device 100 shown in FIG. 1 may be representative of an inflatable penile prosthesis as shown in FIG. 2. In some implementations, the example implantable fluid operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes one or more fluid control devices 215, one or more pressure sensors 216, and other such components. The example inflatable device 200 includes an electronic control system 208 (similar to the example electronic control system 108 described above with respect to FIG. 1) configured to provide for the transfer of fluid between a reservoir 202 (such as the example reservoir 102 described above with respect to FIG. 1) and a fluid receiver 204 (similar to the example fluid receiver or inflatable member 104 described above with respect to FIG. 1) in the form of a pair of inflatable cylinders, via the fluidics components. Fluidics components of the fluid control system 206, and electronic components of the electronic control system 208 may be received in a housing 210. In some implementations, fluidics components of the fluid control system 206, and electronic components of the electronic control system 208 received in the housing 210 may together define an electronically controlled fluid manifold 230 that provides for the electronic control of the flow of fluid between the reservoir 202 and the fluid receiver 204. A first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the fluid receiver 204 in the form of the inflatable cylinders. The electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 may be charged and/or recharged by a power transmission module of the external controller 220, and/or by a power transmission device 250, that is separate from the external controller 220.

The principles to be described herein may be applied to the example implantable fluid operated inflatable device, in the form of the inflatable penile prostheses shown in FIG. 2, and other types of implantable fluid operated inflatable devices that rely on a pump assembly including various fluidics components to provide for the transfer of fluid between the different fluid filled implantable components to achieve inflation, deflation, pressurization, depressurization, deactivation, occlusion, and the like for effective operation. The example implantable fluid operated inflatable device 200 shown in FIG. 2 includes an electronic control system 208 to provide for control of the operation of the respective fluid receivers 204 in the form of cylinders, and the monitoring and control of pressure and/or fluid flow through fluid receivers 204. Some of the principles to be described herein may also be applied to implantable fluid operated inflatable devices that are manually controlled.

As noted above, the electronic control system 208 controlling the flow of fluid between the reservoir 202 and the fluid receiver 204 for inflation, pressurization, deflation, depressurization and the like of the fluid receiver 204 may provide for improved patient control of the inflatable device 200, improved accuracy in operation of the inflatable device 200, improved patient comfort, improved patient safety, and the like. However, in some situations, a size and/or a configuration of the electronic control system 208 and/or the fluid control system 206 (i.e., a size and/or a configuration of the electronically controlled fluid manifold 230 including the electronic control system 208 and the fluid control system 206) may pose a challenge for some patients. Accordingly, in some implementations, the electronically controlled fluid manifold 230 may include a fluid control system 206 having one or more combined pump and valve devices. The use of combined pump and valve devices may reduce a number of active components within the electronically controlled fluid manifold 230, thus reducing the overall size of the electronically controlled fluid manifold 230.

A fluid control system, in accordance with implementations described herein, can include a pump assembly including, for example, one or more pump and valve devices within a fluid circuit of the pump assembly to control the transfer fluid between the fluid reservoir and the fluid receiver. In some examples, the pump assembly including the one or more pump and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump/valve device(s) include piezoelectric elements. In some examples, the pump assembly includes one or more pressure sensing devices in the fluid circuit to provide for relatively precise monitoring and control of fluid flow and/or fluid pressure within the fluid circuit and/or the fluid receiver. A fluid circuit configured in this manner may facilitate the proper inflation, deflation, pressurization, depressurization, and deactivation of the components of the implantable fluid operated device to provide for patient safety and device efficacy.

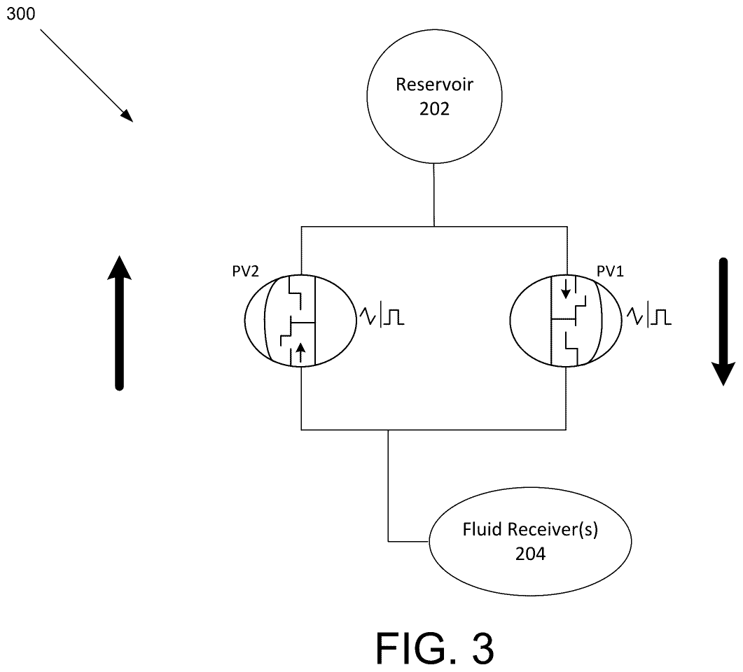

FIG. 3 is a schematic diagram of an example fluidic architecture for an implantable fluid operated inflatable device, according to an aspect. The fluidic architecture shown in FIG. 3 includes combination pump/valves positioned between the reservoir 202 and the fluid receiver 204, to control the flow of fluid between the reservoir 202 and the fluid receiver 204. The fluidic architecture of an implantable fluid operated inflatable device can include other arrangements of fluidic channels, pump(s)/valve(s), pressure sensor(s) and other components than shown in FIG. 3.

In particular, the example fluidic architecture 300 shown in FIG. 3 includes a first fluid control device, or combined pump and valve device, PV1 positioned in a first fluid passageway and controlling the flow of fluid from the reservoir 202 to the fluid receiver 204, and a second fluid control device, or combined pump and valve device, PV2 positioned in a second fluid passageway and controlling the flow of fluid from the fluid receiver 204 to the reservoir 202. The first and second fluid passageways can be conduits through which fluid flows within the fluidic architecture 300 between the reservoir 202 and the receiver(s) 204.

In the example arrangement shown in FIG. 3, the first combined pump and valve device PV1 and the second combined pump and valve device PV2 may be operated in a first mode to inflate or pressurize the fluid receiver 204, and in a second mode to deflate or repressurize the fluid receiver 204. In the first mode of operation, the first combined pump and valve device PV1 may be operable to convey fluid from the reservoir 202 to the fluid receiver 204, while the second combined pump and valve device PV2 remains closed/inoperable to prevent flow of fluid from the fluid receiver 204 towards the reservoir 202 to prevent deflation/depressurization. The first combined pump and valve device PV1 may remain operable to pump fluid to the fluid receiver 204 until a desired pressure is achieved. The first combined pump and valve device PV1 may be closed once the desired pressure is achieved, to maintain the fluid receiver 204 at the desired pressure/inflated state. In the second mode of operation, the second combined pump and valve device PV2 may be operable to convey fluid from the fluid receiver 204 to the reservoir 202, while the first combined pump and valve device PV1 remains closed/inoperable to prevent flow of fluid from the reservoir 202 towards the fluid receiver 204 to prevent inflation/pressurization. The second combined pump and valve device PV2 may remain operable to pump fluid to the reservoir 202 until a desired pressure is achieved at the fluid receiver 204. The second combined pump and valve device PV2 may be closed once the desired pressure is achieved, to maintain the fluid receiver 204 at the desired pressure/in the deflated state.

In the example implantable fluid operated devices described herein, a pressure sensor can be included in the device to monitor and/or measure one or more pressures of fluid in the devices. An electrical signal from the pressure sensor can then be used to control the pressure of the fluid in the device, for example, to optimize a performance of the device or to prevent damage to the device or to a user in whom the devices implanted.

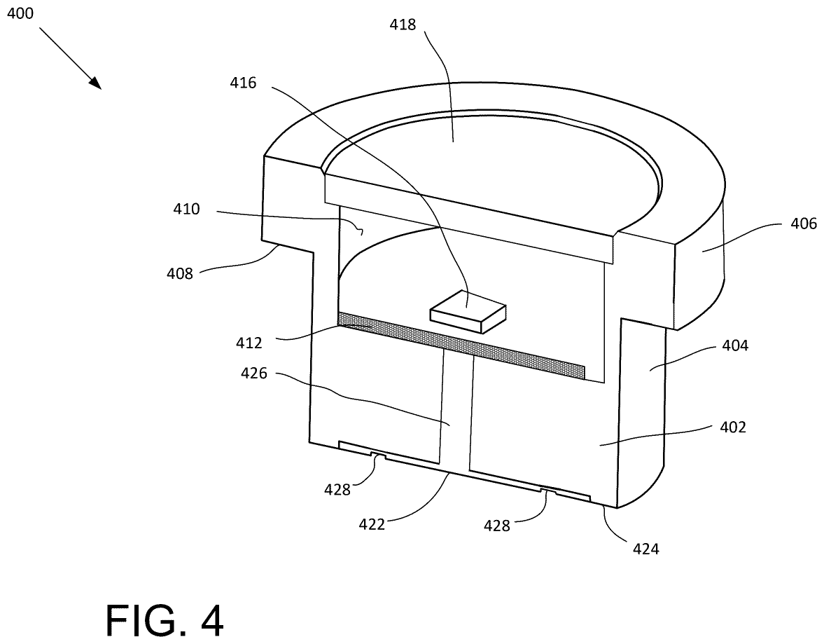

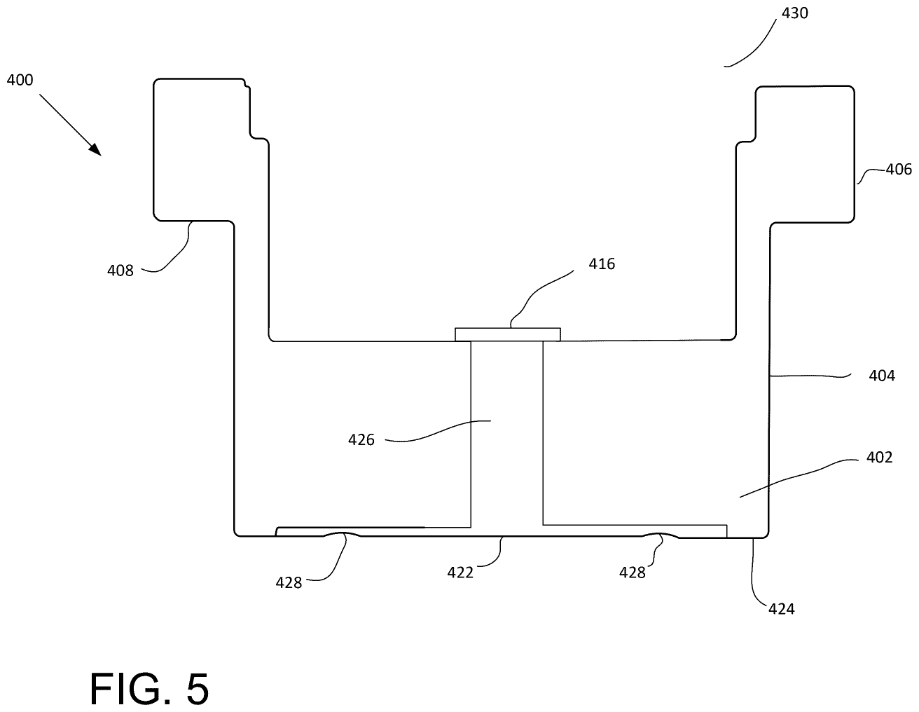

FIG. 4 is a schematic cutaway perspective view of an example pressure sensor 400, and FIG. 5 is a cross-sectional view of the example pressure sensor 400. The pressure sensor can include a metal housing 402, which, in some implementations, can have a generally cylindrical shape, with one or more circular sidewalls 404, 406. In some implementations, the metal housing 402 can be made of titanium or a titanium alloy. In some implementations, the sidewalls 404, 406 may have different diameters. In an implementation in which the sidewalls 404, 406 have different diameters, the metal housing 402 can include a flange 408 that extends radially outward from a diameter of a first sidewall 404. The flange 408 can be configured such that the pressure sensor 400 can fit into a receptacle or recess of a housing of an implantable fluid operated device, and the flange can mechanically couple to, or seat on, a corresponding flange of the housing of the implantable fluid operated device.

The metal housing 402 can define one or more interior cavities within the housing. For example, the metal housing can include an upper cavity 410 that is configured at least for holding electronic components of the pressure sensor. The upper cavity 410 can house a printed circuit board 412 on which electrical circuitry and/or electrical components are connected. For example, the electrical circuitry can include, among other things, a sensor (e.g., a MEMS sensor) and a processor 416 that are connected to the printed circuit board 412.

The pressure sensor 400 can include a top plate 418 that can be fitted onto the metal housing 402 to close the upper cavity 410 after the electrical circuitry is positioned within the upper cavity 410. The top plate 418 can be used to locate and retain electrical connectors that electrically connect components within the housing 402 to components outside the housing. In some implementations, the top plate 418 can hermetically seal against the housing 402, so liquid cannot enter the interior of the housing 402 between the top plate 418 and the housing 402. In some implementations, the top plate 418 can be glued, welded, or otherwise attached to the housing 402. In some implementations, the top plate can be sealed against the housing 402 with a connection that does not rely on a welded joint between the top plate 418 and the housing 402. For example, a flexible O-ring between the top plate 418 and the housing 402 can form the hermetic seal between the top plate 418 and the housing 402. The pressure sensor 400 can include one or more electrical connectors (not shown) that extend through the top plate 418 to receive electrical signals from, and to provide electrical signals to, the electrical circuitry housed within the upper cavity 410 of the pressure sensor.

The pressure sensor 400 also can include a flexible metal diaphragm 422 that is attached to a bottom portion of the metal housing 402. In an example implementation, the flexible metal diaphragm 422 can include an annular stiffening ring 428 that can be stamped into a profile of the diaphragm 422. The flexible metal diaphragm 422 can be made from the same material as the metal housing 402, such as, for example, titanium or titanium alloy and can have a small thickness of, for example, 40 μm or less, 25 μm or less, or 16 μm or less.

In an implementation in which the metal housing 402 includes a cylindrical sidewall 404, the metal housing 402 can include a perimeter rim 424 at a bottom of the cylindrical sidewall 404, and the flexible metal diaphragm 422 can be attached to the perimeter rim. The metal housing 402 of the pressure sensor 400 can additionally define an interior cavity 426 that can be filled with a fluid (e.g., an incompressible silicone oil). When the flexible metal diaphragm is attached to the metal housing 402, fluid in the interior cavity 426 can mechanically and fluidically couple movement of the flexible metal diaphragm 422 to the MEMS sensor on the printed circuit board 412. In this manner, when the pressure sensor 400, or at least a lower portion of the housing 402 and the metal diaphragm 422, is placed into a fluid passageway of a fluidic system, a pressure of fluid in the fluid passageway and outside the pressure sensor 400 on the flexible metal diaphragm 422 can be transmitted to the MEMS sensor. For example, after the interior cavity 426 is filled with fluid, with the flexible metal diaphragm attached to the metal housing 402, electrical signals due to pressure of the fluid in the cavity 426 on the MEMS sensor can be calibrated against known pressures outside the housing 402. Then, variations in pressure of fluid on an outside surface of the flexible metal diaphragm 422 can cause the diaphragm 422 to flex and move toward or away from the MEMS sensor and, because the fluid in the cavity 426 has a low compressibility, the movement of the diaphragm 422 results in movement of a corresponding mechanical element of the MEMS sensor, which is converted to an electrical signal representing a pressure on the outside of the flexible metal diaphragm 422.

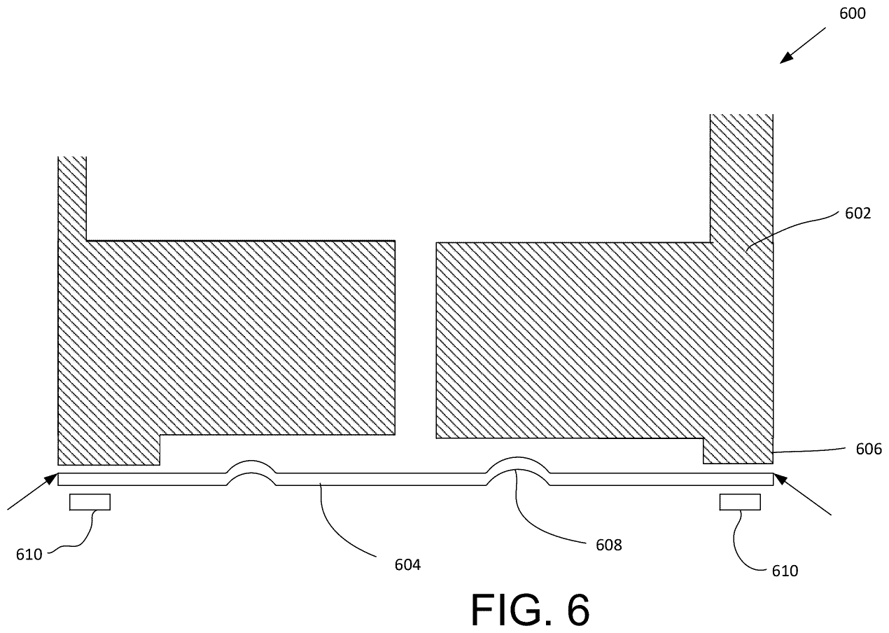

FIG. 6 is a schematic cross-sectional view of an example metal housing 602 and an example flexible metal diaphragm 604 of a pressure sensor 600. The metal housing 602 can have a generally cylindrical shape and can have a perimeter rim 606 with a bottom side. The flexible metal diaphragm 604 can have a generally circular shape with a diameter similar to the diameter of the cylindrical metal housing 602 and with a thickness that is, for example, less than 40 μm, less than 25 μm, or less than 15 μm. The diaphragm 604 can include an annular stiffening ring 608. In some implementations, both the metal housing 602 and the flexible metal diaphragm 604 can include titanium or a titanium alloy. The flexible metal diaphragm 604 can be attached to the metal housing (e.g., to the perimeter rim 606 of the metal housing) by a welding process to create a welded joint between the diaphragm and the housing. The metal diaphragm can be placed in contact with the perimeter rim 606 and then heat can be applied to, or generated at, the contact point (shown by the arrows in FIG. 6) between the diaphragm and the metal housing 602 (e.g., at the perimeter of the diaphragm) to melt the metals so that the diaphragm 604 and the housing 602 fuse at the weld joint. With the thickness of the diaphragm 604 being less than 50 μm, the weld joint may extend through the entire thickness of the diaphragm.

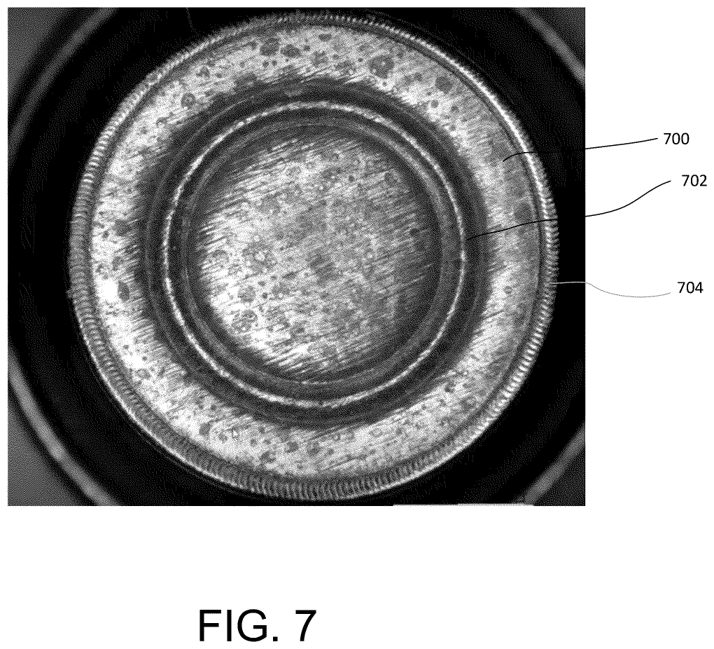

FIG. 7 is a photograph of a flexible titanium diaphragm 700, seen from the bottom, or exterior, side after the diaphragm has been welded to a bottom face of the metal housing. The flexible titanium diaphragm 700 has an annular stiffening ring 702, and the weld joint 704 is clearly visible at the perimeter of the diaphragm.

Referring again to FIG. 6, a consequence of the welding process can be that the heat applied to the metal of the flexible diaphragm 604 during the welding process can alter the microscopic grain structure of the metal in the diaphragm, in particular, by increasing the grain sizes of the metal in the diaphragm. However, because cracks and fissures in the diaphragm 604 can propagate along specific crystallographic planes within the grains, and the flexing of the diaphragm 604 during its designed operation in the pressure sensor can encourage the growth of cracks and fissures, care is taken to ensure that grain sizes in the portion of the diaphragm that flexes during its operation in the pressure sensor are less than 25% of the thickness of the diaphragm. For example, when the thickness of the metal diaphragm is about 40 μm the characteristic metal grain sizes of the flexible portion of the diaphragm can be less than 10 μm. In another example, when the thickness of the metal diaphragm is about 25 μm the characteristic metal grain sizes of the flexible portion of the diaphragm can be less than 6 μm. In another example, when the thickness of the metal diaphragm is about 16 μm the characteristic metal grain sizes of the flexible portion of the diaphragm can be less than 4 μm.

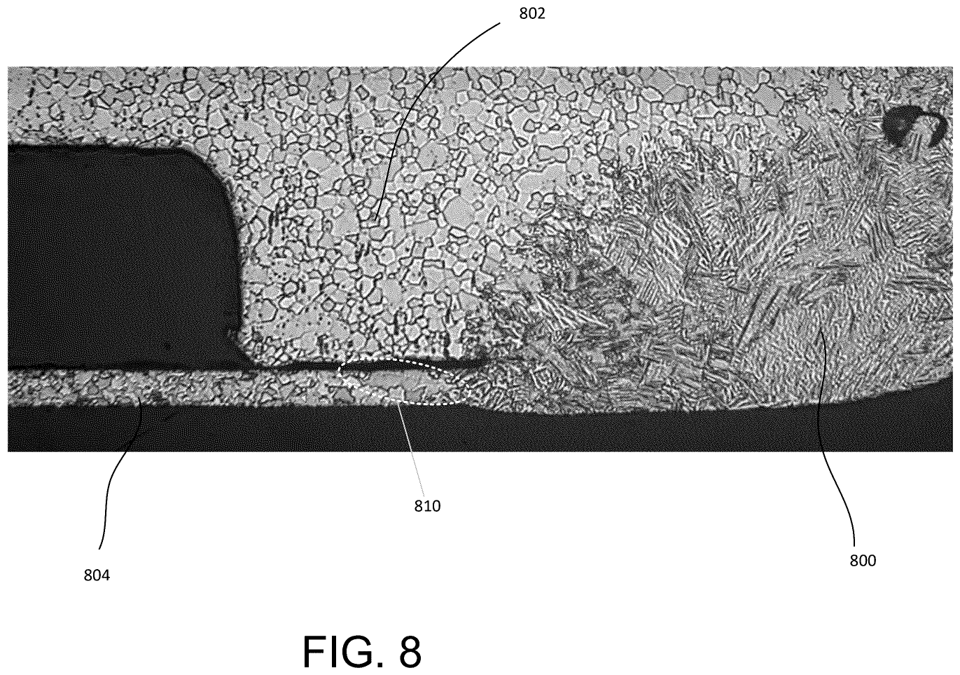

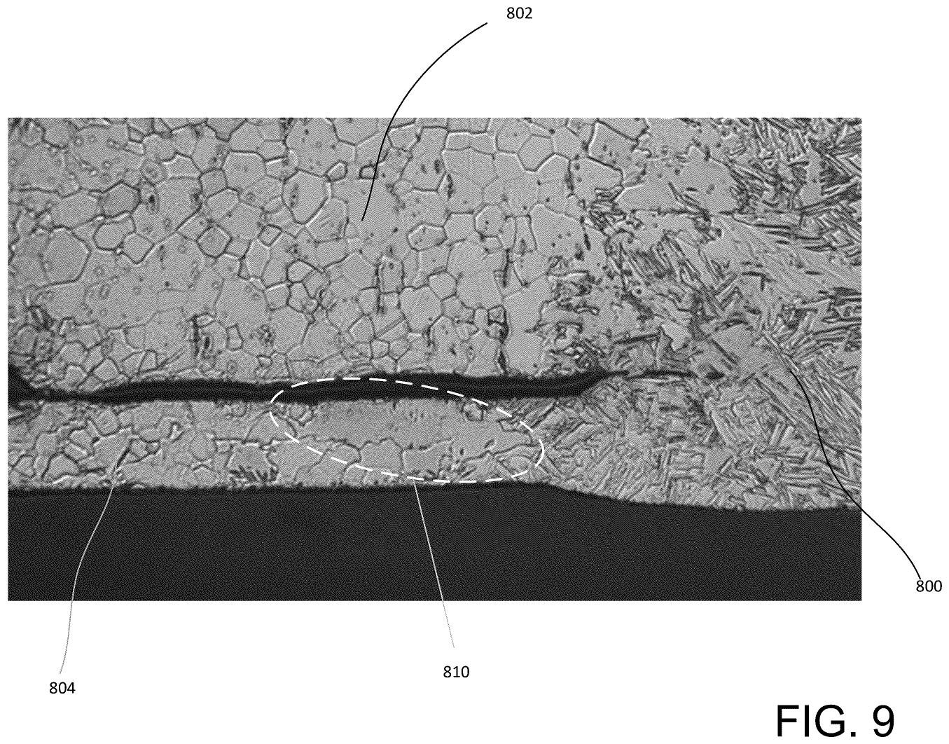

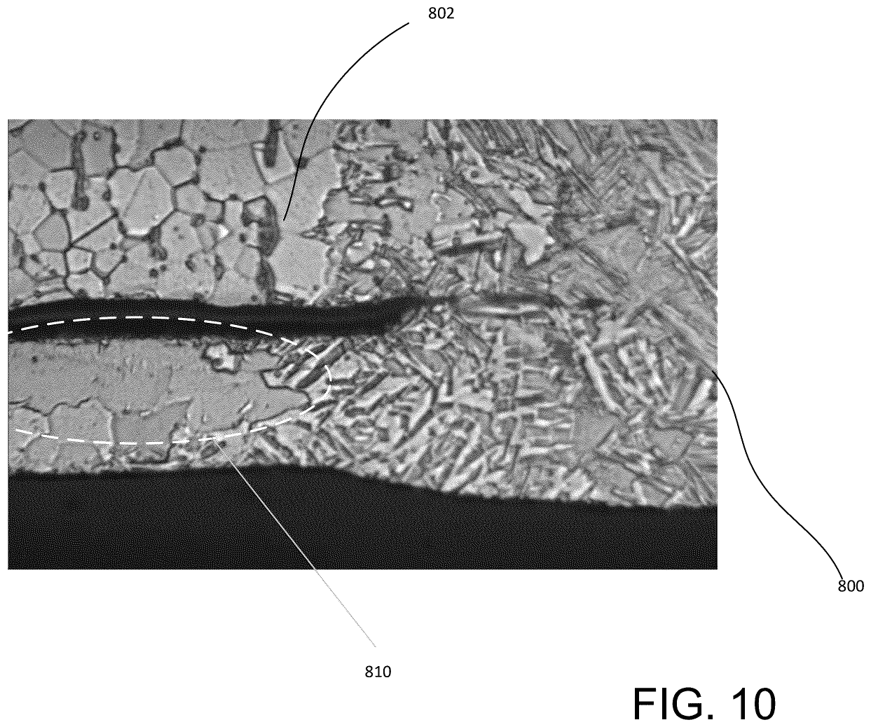

For example, FIG. 8 shows a microscopic image of a weld joint 800 between a flexible titanium diaphragm 804 and a bottom surface of a perimeter rim of a metal housing 802. FIGS. 9 and 10 are enlarged versions of the microscopic image of the weld joint 800 shown in FIG. 8. As seen in FIGS. 8-10, relatively large metal grains exist in the diaphragm adjacent to the weld joint 800, and at least one relatively large metal grain 810 exists in the flexible metal diaphragm 804 in a portion of the diaphragm that is proximate to the weld joint. As seen in FIG. 10, the metal grain 810 has a size in a direction perpendicular to the outside surface of the diaphragm, which is approximately 50-60% of the thickness of the diaphragm. However, the size of metal grains that are farther away from the weld joint 800 are smaller than about 25% of the thickness of the diaphragm. Therefore, to mitigate the increase of metal grain sizes in the flexible portion of the diaphragm 804, techniques are used to prevent or inhibit the propagation of heat from the weld joint 800 to the portion of the flexible metal diaphragm that flexes during operation of the pressure sensor.

Referring again to FIG. 6, an optional heatsink 610 can be placed in contact with the metal diaphragm 604 and/or the perimeter rim 606 of the metal housing 602 to which the metal diaphragm is welded, and, during the welding process, the heatsink can function to conduct heat in the flexible metal diaphragm 604 away from the welded joint. In some implementations, the heatsink 610 can be a conductive metal ring having a diameter that is similar to, and in some cases slightly less than, the diameter of the flexible metal diaphragm. In some implementations, the heatsink 610 can be a conductive piece of metal that is placed in contact with the diaphragm proximate to the welding site and moved along the diaphragm as the weld joint is formed around the perimeter of the diaphragm.

In some implementations, the heatsink 610 can be formed of a metal having a relatively high thermal conductivity and that is dissimilar to the middle of the flexible metal diaphragm 604 and of the metal housing 602. For example, when the diaphragm 604 and the housing 602 are made of titanium or titanium alloy, a copper heatsink 610 would not be welded to the flexible metal diaphragm 604 during the welding process, despite the lower melting point of copper compared to titanium and titanium alloys, so that the copper heatsink could be removed from the structure after the weld is formed between the diaphragm 604 and the metal housing 602. Thus, for example, to prevent the growth of metal grain sizes in the flexible portion of the metal diaphragm 604, the heatsink 610 can conduct heat away from the metal of the diaphragm 604 as the metal is welded to inhibit propagation of heat away from the site of the weld into the flexible portion of the flexible metal diaphragm 604.

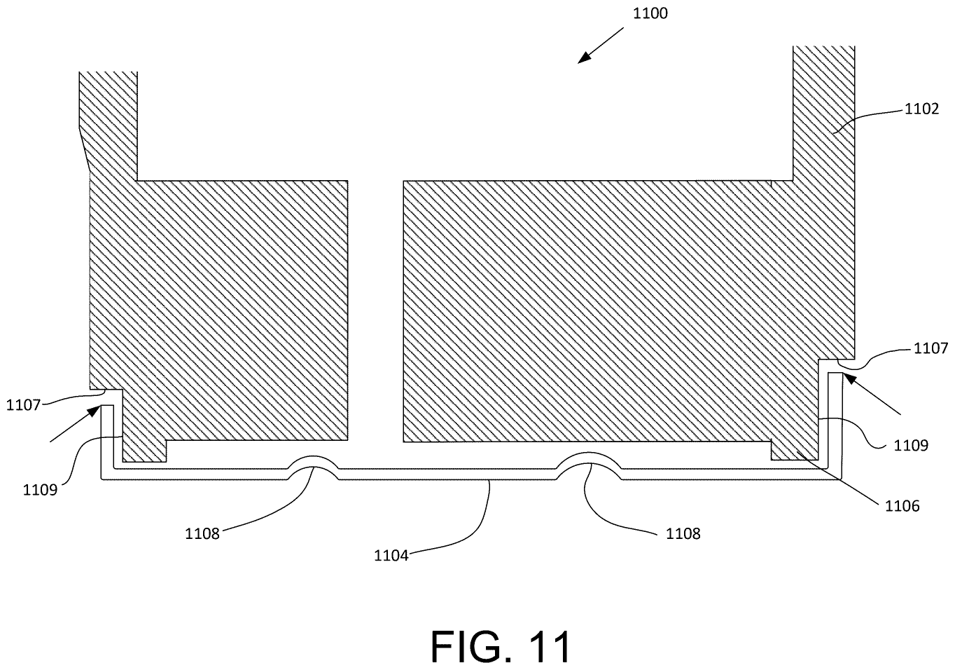

FIG. 11 is a schematic cross-sectional view of another example metal housing 1102 and an example flexible metal diaphragm 1104 of a pressure sensor 1100. The metal housing 1102 can have a generally cylindrical shape and can have a perimeter rim 1106 with a bottom side and a flange 1107 that extends inward from the diameter of the metal housing 1102 above the bottom side of the perimeter rim. The flexible metal diaphragm 1104 can have a generally circular shape with a diameter similar to the diameter of the cylindrical metal housing 1102. However, in contrast to the diaphragm 604, the diameter of the diaphragm 1104 can be slightly larger than the diameter of the cylindrical metal housing 1102, so that a perimeter portion of the diaphragm 1104 can wrap around the bottom side of the perimeter rim 1106 for attachment to the flange 1107 of the metal housing 1102. The flexible metal diaphragm 1104 can have a thickness that is, for example, less than 40 μm, less than 25 μm, or less than 15 μm. The diaphragm 1104 can include an annular stiffening ring 1108. In some implementations, both the metal housing 1102 and the flexible metal diaphragm 1104 can include titanium or titanium alloys. In some implementations, the radius of curvature of the portion of the diaphragm 1104 that bends around the perimeter rim 1106 of the metal housing 1102 can be at least two times the thickness of the diaphragm. In some implementations, the radius of curvature of the portion of the diaphragm 1104 that bends around the perimeter rim 1106 of the metal housing 1102 can be at least five times the thickness of the diaphragm. In some implementations, the radius of curvature of the portion of the diaphragm 1104 that bends around the perimeter rim 1106 of the metal housing 1102 can be at least ten times the thickness of the diaphragm.

The flexible metal diaphragm 1104 can be attached to the metal housing (e.g., to the perimeter rim 1106 of the metal housing and, in some cases, to the sidewall 1109 of the metal housing 1102, where the sidewall is between the flange 1107 and the bottom surface of the perimeter rim 1106) by a welding process to create a welded joint between the diaphragm and the housing. The metal diaphragm can be placed in contact with the perimeter rim 1106 and then heat can be applied to, or generated at, the contact point (shown by the arrows in FIG. 11) between the diaphragm and the metal housing 1102 (e.g., at the perimeter of the diaphragm) to melt the metals so that the diaphragm 1104 and the housing 1102 fuse at the weld joint. With the thickness of the diaphragm 1104 being less than 50 μm, the weld joint may extend through the entire thickness of the diaphragm and may form a weld joint between the perimeter edge of the diaphragm and the flange 1107 of the metal housing 1102 and between the diaphragm and the sidewall 1109 of the metal housing 1102.

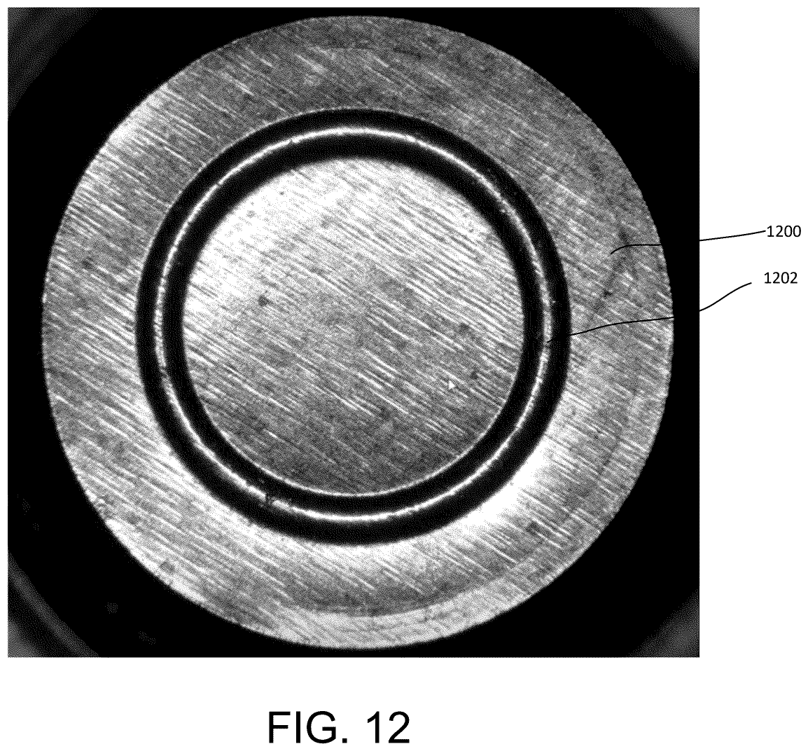

FIG. 12 is a photograph of a flexible titanium diaphragm 1200, seen from a bottom, or exterior, side after the diaphragm has been welded to a flange of a metal housing. The flexible titanium diaphragm 1200 has an annular stiffening ring 1202. Because the weld between the diaphragm and the housing occurs at the flange of the housing, the weld joint is not visible in the bottom view of the diaphragm 1200 seen in FIG. 12.

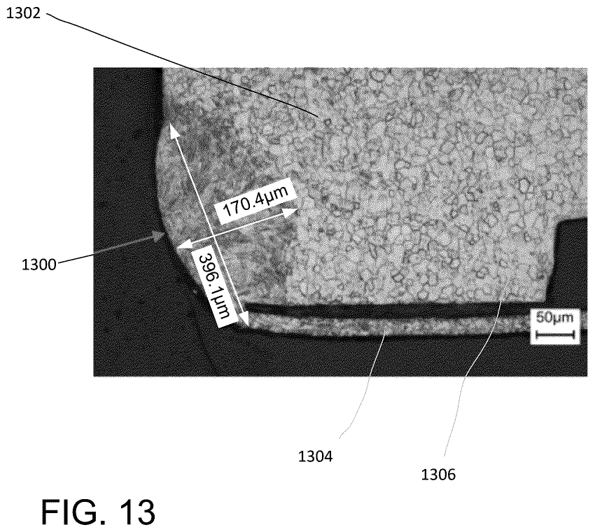

FIG. 13 is a microscopic image of a weld joint 1300 between a flexible titanium diaphragm 1304 and a portion of a metal housing 1302 having a perimeter rim 1306 at a bottom side of the housing. As seen in FIG. 13, by locating the weld joint at the flange of the metal housing, the weld joint 1300 is sufficiently far from the flexible portion of the diaphragm, so that enough heat from the location of the weld joint does not propagate through the metal of the diaphragm to significantly grow the metal grain sizes in the flexible portion of the diaphragm. Therefore, the size of metal grains in the flexible portion of the diaphragm is smaller than about 25% of the thickness of the diaphragm. For example, when the thickness of the metal diaphragm is about 40 μm the characteristic metal grain sizes of the flexible portion of the diaphragm can be less than 10 μm. In another example, when the thickness of the metal diaphragm is about 25 μm the characteristic metal grain sizes of the flexible portion of the diaphragm can be less than 6 μm. In another example, when the thickness of the metal diaphragm is about 16 μm the characteristic metal grain sizes of the flexible portion of the diaphragm can be less than 4 μm.

Although FIG. 13 shows the weld joint 1300 encroaching on the horizontal portion of the metal diaphragm 1304, moving the flange and the weld joint farther away from the horizontal portion of the metal diaphragm and/or changing parameters of the welding process (e.g., using lower power and/or welding times) can ensure that the weld joint and the heat affected zone do not encroach on the horizontal portion of the metal diaphragm. In some implementations, to prevent the growth of metal grain sizes in the flexible portion of the metal diaphragm, the weld joint can be at least 1 mm away from the flexible portion of the metal diaphragm.

In some implementations, the flexible metal diaphragm 1304 can be attached to the metal housing 1302 through a diffusion bonding (e.g., solid-state welding) process in which a surface of the diaphragm 1304 is pressed into contact against a surface of the metal housing at a high pressure to cause the metal surfaces to intersperse themselves and to form a metallurgical joint between the diaphragm and the housing. The diffusion bonding process can be carried out at temperatures that are significantly lower than the melting point of titanium or typical temperatures used in a welding process, and the lower temperatures can reduce the extent of, or eliminate, any heat affected zones that are formed at the joint between the diaphragm and the metal housing.

For example, referring again to FIG. 6, the diaphragm 604 can be pressed against the bottom surface of the perimeter rim 606 of the metal housing 602. For example, the diaphragm 604 and the metal housing 602 can be placed between bolsters of a high-pressure press, and then press can be used to press the diaphragm 604 against the bottom surface of the perimeter rim 606 of the metal housing 602. In some implementations, the pressure can be at least 60 PSI. In some implementations, the pressure can be at least 200 PSI. In some implementations, the pressure can be applied for at least 10 minutes, for at least 30 minutes, for at least 60 minutes, or for at least 120 minutes.

To accomplish a strong and reliable metallurgical joint between the diaphragm 604 and the housing 602 from a diffusion bonding process, contaminant materials must be removed from the surfaces of the metals to be bonded. In some implementations, the diaphragm 604 and the metal housing 602 can be placed in a vacuum chamber that is then evacuated and then the diaphragm can be pressed against the bottom surface of the perimeter rim 606 of the metal housing 602 to form the diffusion bond between the metals. In some implementations, the diaphragm 604 and the metal housing 602 can be heated before they are pressed together, where heating the diaphragm 604 and the metal housing 602 to a temperature of more than about 850° C. can dissolve a titanium oxide layer on the surfaces of the diaphragm and the housing. In some implementations, the diaphragm 604 and the metal housing 602 can be pressed together when their temperatures are more than 850° C. but less than 1200° C. In some implementations, the diaphragm 604 and the metal housing 602 can be pressed together when their temperatures are more than 850° C. but less than 1050° C. In some implementations, after the vacuum chamber is evacuated of hydrogen and oxygen, a noble gas (e.g., argon) atmosphere can be introduced into the vacuum chamber, and the diffusion bonding process can be carried out in the noble gas environment.

Referring again to FIG. 11, the diaphragm 1104 can be squeezed against an outer surface of sidewall 1109 of the metal housing 1102. For example, a collar can be placed aground the sidewall 1109, with the diaphragm between the collar and the sidewall, and then collar can squeeze the diaphragm 1104 against the sidewall 1109, for example, at a pressure of at least 60 PSI or of at least 200 PSI.

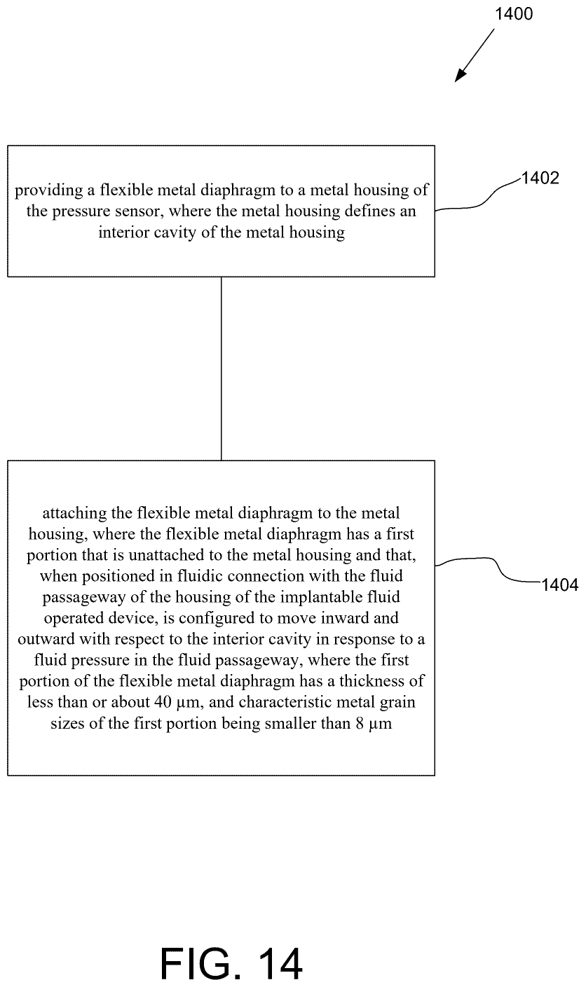

FIG. 14 is a flowchart of an example process 1400 for making a pressure sensor to be positioned in fluidic connection with a fluid passageway of a housing of an implantable fluid operated device. The process 1400 includes providing a flexible metal diaphragm to a metal housing of the pressure sensor (1402), where the metal housing defines an interior cavity of the metal housing. The process further includes attaching (e.g., welding, diffusion bonding, etc.) the flexible metal diaphragm to the metal housing, where the flexible metal diaphragm has a first portion that is unattached to the metal housing and that, when positioned in fluidic connection with the fluid passageway of the housing of the implantable fluid operated device, is configured to move inward and outward with respect to the interior cavity in response to a fluid pressure in the fluid passageway (1404). The first portion of the flexible metal diaphragm has a thickness of less than or about 40 μm, and characteristic metal grain sizes of the first portion being smaller than 8 μm.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated device, comprising:
a fluid reservoir;
a fluid receiver; and
a fluid control system configured to control fluid flow between the fluid reservoir and the fluid receiver, the fluid control system including:
a housing including a fluidic architecture defining one or more fluid passageways within in the housing;
at least one pump positioned in fluidic connection with at least one of the one or more fluid passageways, the at least one pump being configured to pump fluid from the fluid reservoir to the fluid receiver;
a pressure sensor positioned in fluidic connection with at least one of the one or more fluid passageways, the pressure sensor including:
a metal housing including one or more interior cavities;
electrical circuitry configured for converting a pressure into an electrical signal;
a flexible metal diaphragm attached to the metal housing and having a first portion positioned between an interior cavity of the one or more interior cavities and a fluid passageway and the first portion being configured to move inward and outward with respect to an interior cavity in response to a fluid pressure in the fluid passageway, the first portion of the flexible metal diaphragm having a thickness of less than 40 μm, and characteristic metal grain sizes of the first portion being smaller than 10 μm.

2. The implantable fluid operated device of claim 1, wherein the metal housing is a titanium housing and wherein the flexible metal diaphragm is a flexible titanium diaphragm.

3. The implantable fluid operated device of claim 2, wherein the flexible metal diaphragm is attached to the metal housing by a welded joint between the flexible metal diaphragm and the metal housing.

4. The implantable fluid operated device of claim 3, wherein the welded joint between the flexible metal diaphragm and the metal housing is made at a second portion of the flexible metal diaphragm that is more than 1 mm away, along a surface of the flexible metal diaphragm, from any part of the first portion of the flexible metal diaphragm.

5. The implantable fluid operated device of claim 4, wherein the second portion of the flexible metal diaphragm is non-parallel to the first portion of the flexible metal diaphragm.

6. The implantable fluid operated device of claim 2, wherein the flexible metal diaphragm is attached to the metal housing by a diffusion bonded joint between the flexible metal diaphragm and the metal housing.

7. The implantable fluid operated device of claim 6, wherein the diffusion bonded joint between the flexible metal diaphragm attached to the metal housing is made at a second portion of the flexible metal diaphragm that is more than 1 mm away, along a surface of the flexible metal diaphragm, from any part of the first portion of the flexible metal diaphragm.

8. The implantable fluid operated device of claim 7, wherein the second portion of the flexible metal diaphragm is non-parallel to the first portion of the flexible metal diaphragm.

9. The implantable fluid operated device of claim 7, wherein the thickness of the first portion of the flexible metal diaphragm is less than 25 μm, and metal grain sizes of the first portion are smaller than 6 μm.

10. The implantable fluid operated device of claim 7, wherein the thickness of the first portion of the flexible metal diaphragm is less than 16 μm, and metal grain sizes of the first portion are smaller than 4 μm.

11. The implantable fluid operated device of claim 1, wherein the one or more interior cavities include at least one fluid-filled cavity that is fluidically coupled to the flexible metal diaphragm and to the electrical circuitry, wherein the electrical circuitry is configured for converting a displacement of the flexible metal diaphragm into the electrical signal.

12. A method of making a pressure sensor to be positioned in fluidic connection with a fluid passageway of a housing of an implantable fluid operated device, the method comprising:
providing a flexible metal diaphragm to a metal housing of the pressure sensor, wherein the metal housing defines an interior cavity of the metal housing; and
attaching the flexible metal diaphragm to the metal housing, the flexible metal diaphragm having a first portion that is unattached to the metal housing and that, when positioned in fluidic connection with the fluid passageway of the housing of the implantable fluid operated device, is configured to move inward and outward with respect to the interior cavity in response to a fluid pressure in the fluid passageway, the first portion of the flexible metal diaphragm having a thickness of less than 40 μm, and characteristic metal grain sizes of the first portion being smaller than 10 μm.

13. The method of claim 12, wherein the metal housing is a titanium housing and wherein the flexible metal diaphragm is a flexible titanium diaphragm.

14. The method of claim 13, wherein attaching the flexible metal diaphragm to the metal housing includes welding the flexible metal diaphragm to the metal housing.

15. The method of claim 14, wherein welding the flexible metal diaphragm to the metal housing includes welding the flexible metal diaphragm to the metal housing at a second portion of the flexible metal diaphragm that is more than 1 mm away, along a surface of the flexible metal diaphragm, from any part of the first portion of the flexible metal diaphragm.

16. The method of claim 15, wherein the second portion of the flexible metal diaphragm is non-parallel to the first portion of the flexible metal diaphragm.

17. The method of claim 13, wherein attaching the flexible metal diaphragm to the metal housing includes diffusion bonding the flexible metal diaphragm to the metal housing.

18. The method of claim 17, wherein diffusion bonding the flexible metal diaphragm to the metal housing includes diffusion bonding the flexible metal diaphragm to the metal housing at a second portion of the flexible metal diaphragm that is more than 1 mm away, along a surface of the flexible metal diaphragm, from any part of the first portion of the flexible metal diaphragm.

19. The method of claim 18, wherein the second portion of the flexible metal diaphragm is non-parallel to the first portion of the flexible metal diaphragm.

20. The method of claim 13, further comprising filling the interior cavity with fluid that is fluidically coupled to the flexible metal diaphragm and to electrical circuitry in the pressure sensor.
